(19) United States
(12) Patent Application Publication
(10) Pub. No.: US 2023/0204560 A1
(43) Pub. Date: Jun. 29, 2023

(54) METHOD, SYSTEM AND MEDIUM FOR PAPERMAKING QUALITY EVALUATION

(71) Applicant: AKTIEBOLAGET SKF, GOTEBORG (SE)

(72) Inventors: Kaihuan ZHANG, Hangzhou (CN); Gang CHENG, Shanghai (CN)

(21) Appl. No.: 17/986,268

(22) Filed: Nov. 14, 2022

(30) Foreign Application Priority Data
Dec. 23, 2021 (CN) 202111588571.8

(51) Int. Cl. G01N 33/34; G06K 9/62

(52) U.S. Cl. CPC G01N 33/34; G06K 9/6289; G06K 9/629; G06K 9/6232

(57) ABSTRACT

A method for evaluating papermaking quality includes determining an evaluation target related to papermaking quality and for each evaluation target: acquiring target working condition data, target condition monitoring data and target papermaking quality data; preprocessing the acquired data; performing data integration on the preprocessed target working condition data, the preprocessed target condition monitoring data, and the preprocessed target papermaking quality data to obtain an integrated data set; performing feature extraction on data in the integrated data set according to types and characteristics of the data; establishing a paper quality analysis model based on the target feature data set; and evaluating the corresponding evaluation target and generating a quality health evaluation result of the corresponding evaluation target based on the papermaking quality analysis model. Also obtaining a comprehensive papermaking quality evaluation result based on the quality health evaluation result of the at least one evaluation target.

### CROSS-REFERENCE

This application claims priority to Chinese patent application no. 202111588571.8 filed on Dec. 23, 2021, the contents of which are fully incorporated herein by reference.

### TECHNICAL FIELD

The present disclosure relates to a field of papermaking, and in particular to a method, system and medium for papermaking quality evaluation.

### BACKGROUND

At present, commonly used evaluation and control of papermaking quality is mainly through paper inspection according to an operator's hand feel during a papermaking process, or papermaking quality is evaluated by quality testing and statistical analysis in laboratory after producing a certain unit (e.g., a roll, a batch, etc.) of paper products. In other words, current quality evaluation are subjective, offline, or a posteriori.

Although some online paper monitoring and evaluation methods have recently emerged, such as a combination of image monitoring and image morphological analysis, these methods mainly focus on a single quality indicator related to physical structure of papers or topography of paper surfaces. In addition, these methods need to process images, which requires a system to have high-speed computing power and storage capacity to satisfy online evaluation.

Therefore, there is a need to develop an improved technology for evaluating papermaking quality, which can realize online, comprehensive and multi-dimensional evaluation of papermaking quality, while avoiding the requirement for high storage capacity and high computing power of the computing system brought about by complex computing.

### SUMMARY OF THE DISCLOSURE

According to an aspect of the present disclosure, a method for evaluating papermaking quality is provided, comprising: determining at least one evaluation target related to papermaking quality; for each evaluation target, acquiring target working condition data, target condition monitoring data and target papermaking quality data related to the corresponding evaluation target; preprocessing the acquired target working condition data, target condition monitoring data and target papermaking quality data to obtain preprocessed target working condition data, preprocessed target condition monitoring data and preprocessed target papermaking quality data; performing data integration on the preprocessed target working condition data, the preprocessed target condition monitoring data, and the preprocessed target papermaking quality data to obtain an integrated data set; performing feature extraction on data in the integrated data set according to types and characteristics of the data based on the integrated data set to obtain a target feature data set; establishing a paper quality analysis model for evaluation of the corresponding evaluation target based on the target feature data set; and evaluating the corresponding evaluation target and generating a quality health evaluation result of the corresponding evaluation target based on the papermaking quality analysis model. The method further comprises: obtaining a comprehensive papermaking quality evaluation result based on the quality health evaluation result of the at least one evaluation target.

In some embodiments, for each evaluation target, performing feature extraction on data in the integrated data set according to types and characteristics of the data based on the integrated data set to obtain a target feature data set includes: extracting features of working condition data in the integrated data set to obtain working condition features; extracting features of condition monitoring data in the integrated data set to obtain condition monitoring features; extracting features of papermaking quality data in the integrated data set to obtain papermaking quality features; obtaining the target feature data set based on the working condition features, the condition monitoring features and the papermaking quality features.

In some embodiments, for each evaluation target, obtaining the target feature data set based on the working condition features, the condition monitoring features and the papermaking quality features includes: obtaining fused feature data by feature fusion processing based on the working condition features, the condition monitoring features and the papermaking quality features, and generating the target feature data set based on the fused feature data.

In some embodiments, for each evaluation target, establishing a paper quality analysis model for evaluation of the corresponding evaluation target based on the target feature data set includes: establishing a quality anomaly detection model for detecting quality anomaly of the corresponding evaluation target, based on the target feature data set; establishing a quality level classification model for classifying a quality level of the corresponding evaluation target, based on the target feature data set; and establishing a quality indicator prediction model for predicting a quality indicator of the corresponding evaluation target, based on the target feature data set.

In some embodiments, for each evaluation target, evaluating the corresponding evaluation target and generating a quality health evaluation result of the corresponding evaluation target based on the papermaking quality analysis model includes: detecting the quality anomaly of the corresponding evaluation target and generating a quality anomaly detection result based on an output of the quality anomaly detection model; classifying the quality level of the corresponding evaluation target and generating a quality level classification result based on an output of the quality level classification model; predicting the quality indicator of the corresponding evaluation target and generating a quality indicator prediction result based on an output of the quality indicator prediction model; and generating the quality health evaluation result of the corresponding evaluation target based on at least one of the quality anomaly detection result, the quality level classification result, and the quality indicator prediction result.

In some embodiments, for each evaluation target, preprocessing the acquired target working condition data, target condition monitoring data and target papermaking quality data comprises performing at least one of: data deduplication processing, data noise reduction processing, data encoding processing, and data filtering processing.

In some embodiments, for each evaluation target, performing data integration on the preprocessed target working condition data, the preprocessed target condition monitoring data, and the preprocessed target papermaking quality data comprises: performing at least one of synchronization, alignment and correction processing on the preprocessed target working condition data, the preprocessed target condition monitoring data, and the preprocessed target papermaking quality data.

In some embodiments, the at least one evaluation target includes at least one component of a papermaking machine.

According to another aspect of the present disclosure, a system for evaluating papermaking quality is provided. The system comprises: an evaluation target determination module configured to determine at least one evaluation target related to papermaking quality; a data acquisition module configured to, for each evaluation target, acquire target working condition data, target condition monitoring data and target papermaking quality data related to the corresponding evaluation target; a data preprocessing module configured to, for each evaluation target, preprocess the acquired target working condition data, target condition monitoring data and target papermaking quality data to obtain preprocessed target working condition data, preprocessed target condition monitoring data and preprocessed target papermaking quality data; a data integration module configured to, for each evaluation target, perform data integration on the preprocessed target working condition data, the preprocessed target condition monitoring data, and the preprocessed target papermaking quality data to obtain an integrated data set; a feature extraction module configured to, for each evaluation target, perform feature extraction on data in the integrated data set according to types and characteristics of the data based on the integrated data set to obtain a target feature data set; an analysis model establishment module configured to, for each evaluation target, establish a paper quality analysis model for evaluation of the corresponding evaluation target based on the target feature data set; a target evaluation module configured to, for each evaluation target, evaluate the corresponding evaluation target and generate a quality health evaluation result of the corresponding evaluation target based on the papermaking quality analysis model; and a papermaking quality evaluation module configured to obtain a comprehensive papermaking quality evaluation result based on the quality health evaluation result of the at least one evaluation target.

According to another aspect of the present disclosure, a computer-readable storage medium having instructions stored thereon is provided, which are executed by a computer to realize the above-mentioned method for evaluating papermaking quality.

### BRIEF DESCRIPTION OF THE DRAWINGS

The system may be better understood with reference to the following description taken in conjunction with accompanying drawings. Components in the drawings are not to scale, emphasis instead being placed upon illustrating principles of the present disclosure. Furthermore, in the drawings, similar or identical reference numerals represent similar or identical elements.

FIG. 1 is a flow chart of a method for papermaking quality evaluation according to one or more embodiments of the present disclosure.

FIG. 2 is a modular framework diagram of a method or system for papermaking quality evaluation, decision and optimization according to one or more embodiments of the present disclosure.

FIGS. 3A-3D are exemplary quality anomaly degree trend diagrams of the method and system according to the present disclosure, which includes quality anomaly degree trend diagrams of four papermaking processes.

FIG. 4 is a confusion matrix of quality level classification results for a plurality of papermaking processes during testing of the method and system of the present disclosure.

FIG. 5 schematically illustrates quality indicator prediction results for a plurality of papermaking processes during testing of the method and system of the present disclosure.

FIG. 6 is a schematic diagram of fitting and regression performance of the quality indicator prediction of FIG. 5.

FIG. 7 is a radar chart of an exemplary paper quality health evaluation drawn based on partial health evaluation results obtained using the method and system for papermaking quality evaluation of the present disclosure.

### DETAILED DESCRIPTION

It should be understood that the following description of embodiments is given for purposes of illustration only and not limitation. Exemplary division of functional blocks, modules or units shown in the drawings should not be construed as implying that these functional blocks, modules or units must be implemented as physically separate units. The functional blocks, modules or units shown or described may be implemented as separate units, circuits, chips, functions, modules or circuit elements. One or more functional blocks or units may also be implemented in a common circuit, chip, circuit element or unit.

Although the present disclosure makes various references to certain modules in a system according to an embodiment of the present disclosure, any number of different modules may be used and run on user terminals and/or servers. The modules are illustrative only, and different aspects of the system and method may use different modules. The modules can also be run on any device, interface or interface system connected to any system used for data collection and monitoring to obtain data from the system. The method of the present disclosure may be performed using a microprocessor, an Application Specific Integrated Circuit (ASIC), a System on a Chip (SoC), a computing device, a portable mobile computing device (e.g., a tablet or a cellphone), or the like. The processor may be configured to perform the various method steps discussed below.

Flow charts are used in the present disclosure to illustrate operations performed by a system according to one or more embodiments of the present disclosure. It should be understood that preceding or following operations are not necessarily performed in exact order. Rather, various steps may be processed in reverse order or concurrently, as desired. At the same time, other operations may be added to these procedures, or a step or steps may be removed from these procedures.

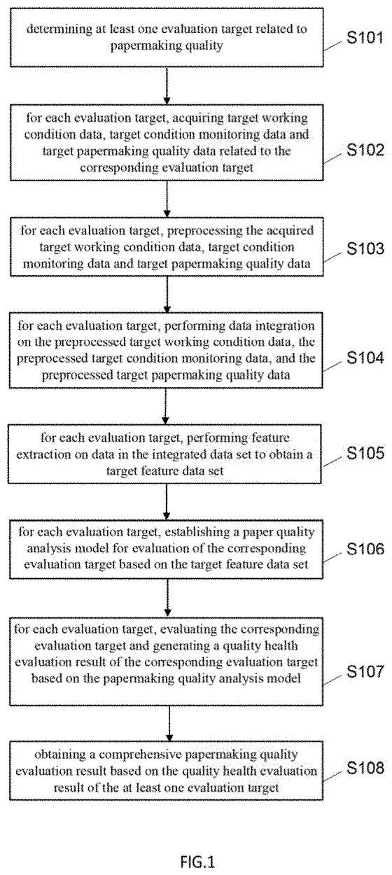

FIG. 1 schematically illustrates a flow chart of a method for papermaking quality evaluation according to one or more embodiments of an aspect of the present disclosure.

Referring to FIG. 1, at S101, at least one evaluation target related to papermaking quality is determined. The at least one evaluation target may include at least one component of a papermaking machine that directly or indirectly has a major impact on papermaking quality, for example, cutters, bearings, and the like.

At S102, for each evaluation target, target working condition data, target condition monitoring data and target papermaking quality data related to the corresponding evaluation target are acquired.

It should be understood that the data herein (target working condition data, target condition monitoring data and target papermaking quality data) may be analog data, or may also be digital data, for example. The data may include signal data such as voltage, current, power, vibration, temperature, and the like. It may be understood that different types of data have different characteristics. The embodiments of the present disclosure are not limited by data types of the data.

The working condition data may mainly include data information that reflects a real-time processing status of the evaluation target or a specific processing of the evaluation target. For example, the working condition data may include time stamp, machine speed, type of process, load, Yankee surface, coating, blade grade, and the like. It should be understood that the embodiments of the present disclosure are not limited by a specific composition and type of the above-mentioned working condition data. In practical applications, the working condition data need to be acquired may also be determined according to actual requirements and actual application scenarios.

The condition monitoring data may be data obtained by monitoring the evaluation target or a specific process of the evaluation target. For example, the condition monitoring data may include blade angle, vibration, temperature, humidity, and the like. It should be understood that the embodiments of the present disclosure are not limited by a specific composition and type of the above-mentioned condition monitoring data. In practical applications, the condition monitoring data need to be acquired may also be determined according to actual requirements and actual application scenarios.

The papermaking quality data may be historical data reflecting quality of paper products produced. For example, papermaking quality data may be acquired and recorded by an inspector's hand feel or post-roll inspection (e.g., laboratory inspection).

In addition, specific sources and acquisition ways of the working condition data, condition monitoring data, and papermaking quality data related to the papermaking evaluation target of the present disclosure may be diverse. For example, the target working condition data related to the evaluation target may be directly obtained from a control system, a working system or other externally connected systems or servers (such as data acquisition and monitoring systems) of the papermaking machine according to a predetermined sampling frequency, or may also be obtained from other sources or in other ways. For example, the condition monitoring data related to the papermaking quality of the evaluation target may be acquired from various sensors disposed around the evaluation target according to a predetermined sampling frequency. In addition, for example, quality historical data on papermaking quality may be collected from a control system, a working system, or other externally connected systems. The papermaking quality data may also be acquired by manual recording, sampling inspection and laboratory analysis according to actual needs. The papermaking quality data may only be used as training data for subsequent establishment of relevant papermaking quality analysis models and evaluation models. It should be understood that the working condition data, condition monitoring data, and papermaking quality data related to the papermaking quality evaluation of the present disclosure may also be obtained in other ways. The embodiments of the present disclosure are not limited by their specific sources and acquisition ways.

At S103, for each evaluation target, the acquired target working condition data, the target condition monitoring data and the target papermaking quality data may be preprocessed to obtain preprocessed target working condition data, preprocessed target condition monitoring data and preprocessed target papermaking quality data.

The process of preprocessing the above-obtained target working condition data, target condition monitoring data and target papermaking quality data may include using various types of algorithms to process the data according to characteristics of the data, so as to filter out currently required valid data, reduce and suppress invalid data and improve data quality. In some embodiments, preprocessing the target operating condition data, the target condition monitoring data, and the target papermaking quality data may include performing, according to types and characteristics of the data, at least one of: data deduplication, data noise reduction processing, data encoding processing, and data filtering processing.

Data deduplication is intended to remove duplicate data. For example, duplicate data may be retrieved and removed based on data such as timestamps, process numbers, and the like.

Data noise reduction processing is intended to remove outliers in the data and realize optimization of the data. For example, methods such as distance-based detection, statistics-based detection, distribution-based outlier detection, density clustering detection, boxplot detection and the like may be used to perform noise reduction on signal data to remove outliers in the data.

Data encoding processing is intended to use different encoding schemes to process data formats as required, so as to obtain encoded data. For example, a required target data format may be determined according to modeling, analysis and evaluation, and the data may be encoded accordingly based on the target data format to facilitate subsequent processing.

Data filtering processing is intended to identify and remove noise in the data and improve a contrast of valid feature information in the data. For example, data filtering may be implemented by using a weighted average filter, a median filter, a Gaussian filter, a Wiener filters, and other methods.

The above only exemplifies several specific processing methods that the preprocessing may include. It should be understood that other preprocessing methods may also be selected according to actual needs. In addition, according to types and characteristics of the data, one or more of the above-mentioned preprocessing methods may be selected to perform preprocessing of the data.

For example, the preprocessed working condition data, the preprocessed condition monitoring data, and the preprocessed papermaking quality data may be respectively recorded as different data subsets according to the above data categories, for example, as data subsets D_{cond}, D_{como}, D_{quality}.

At S104, for each evaluation target, data integration may be performed on the preprocessed target working condition data, the preprocessed target condition monitoring data, and the preprocessed target papermaking quality data to obtain an integrated data set.

In some embodiments, performing data integration on the preprocessed target working condition data, the preprocessed target condition monitoring data, and the preprocessed target papermaking quality data to obtain an integrated data set may include: performing at least one of synchronization, alignment and data correction processing on the preprocessed target working condition data, the preprocessed target condition monitoring data, and the preprocessed target papermaking quality data.

For example, the synchronization, alignment and correction of the preprocessed multi-source data, D_{cond}, D_{como}, D_{quality}, may be completed by using a plurality of algorithms such as interpolation and translation based on a standard clock source, and then a full dataset D for monitoring, evaluation and optimization of papermaking quality is obtained.

Specifically, for example, when the working condition data, condition monitoring data and papermaking quality data related to the evaluation target are acquired by periodic sampling, due to different sampling frequencies selected and different starting times of the sampling process, the acquired working conditions data, condition monitoring data and papermaking quality data, for example, have different time axis starting points, and their respective durations are different. It is also possible that some data is missing or significantly inaccurate due to anomalies in the sampling process, resulting in incomplete data content in the spatial dimension, discontinuous data and inconsistent time series in the time dimension of the acquired original data. In this case, for example, the data may be processed in the time dimension based on a standard clock source, so as to realize synchronization and alignment between multi-source data. At the same time, various algorithms such as interpolation algorithm and translation algorithm may be used to correct and complete data values (i.e., processing in the spatial dimension), so as to obtain the integrated full data set for monitoring and evaluation of papermaking quality.

At S105, for each evaluation target, feature extraction is performed on data in the integrated data set according to types and characteristics of the data based on the integrated data set to obtain a target feature data set.

In some embodiments, the processing of feature extraction may include: extracting features of the working condition data in the data set based on the integrated data set to obtain working condition features; extracting features of the condition monitoring data in the data set based on the integrated data set to obtain condition monitoring features; extracting features of the papermaking quality data in the data set based on the integrated data set to obtain papermaking quality features; and obtain the target feature data set related to the evaluation target based on the working condition features, the condition monitoring features, the papermaking quality features.

The following extraction processes may be performed in any order or in parallel: extracting features of the working condition data in the data set, extracting features of the condition monitoring data in the data set, and extracting features of the papermaking quality data in the data set. In addition, the above feature extraction processes may adopt a plurality of feature extraction methods, and each extraction process may adopt the same extraction method or different extraction methods. An exemplary feature extraction method for the above-described feature extraction processes is illustrated below. For example, depending on actual needs, data extraction of the data set may include, for example, time domain feature extraction, frequency feature extraction, time-frequency domain feature extraction, waveform feature extraction, and the like.

Time-domain feature extraction refers to extracting time-domain features of data (such as acquired signals), including but not limited to mean, variance, standard deviation, maximum value, minimum value, root mean square, peak-to-peak value, skewness, kurtosis, waveform index, pulse index, margin index, and the like. Frequency feature extraction refers to extracting frequency features of data, including but not limited to mean square frequency, frequency variance, frequency band energy, and the like. Time-frequency domain feature extraction refers to extracting time-frequency domain features of data, including but not limited to frequency band energy or time domain characteristics of signals after wavelet decomposition or empirical mode decomposition. Waveform feature extraction refers to extracting waveform features of data, which, for example, when the data is a collected signal, includes but is not limited to the area enclosed by the signal waveform, maximum/minimum derivatives, rising edges, falling edges, and the like.

As for formation of the feature data set, for example, the feature data set may be formed by directly adopting the working condition features, the condition monitoring features, and the papermaking quality features. Alternatively, the working condition features, the condition monitoring features, and the papermaking quality features may be further processed, and the feature data set is obtained based on results of the processing. The embodiments of the present disclosure are not limited by a specific generation method and contents of the feature data set.

In some embodiments, for each evaluation target, obtaining the target feature data set based on the working condition features, the condition monitoring features, and the papermaking quality features may further include: obtaining fused feature data by feature fusion processing based on the working condition features, the condition monitoring features and the papermaking quality features, and generating the target feature data set based on the fused feature data.

For example, based on the feature data set obtained by extraction, feature fusion of different levels and dimensions may be performed to obtain an effective multi-dimensional feature vector. For example, the multi-dimensional feature vector may be expressed as F={Fᵢ|i=1, 2, . . . , k}, where k represents the dimension after feature extraction and fusion. F includes fused features of the working condition features, condition monitoring features and papermaking quality features. The extracted and fused features herein may be features that are used for papermaking quality evaluation, are sensitive to a papermaking process status, and are not easily affected by disturbances caused by working conditions. The fused features can correlate the working condition features, condition monitoring features and papermaking quality features of the evaluation target, so as to evaluate the papermaking quality in a more comprehensive and multi-dimensional way.

For example, the method of feature layer deep fusion may be used, that is, fusing the extracted original features in different dimensions based on distance algorithm, similarity algorithm, weighted average algorithm, principal component analysis algorithm, etc., and obtaining fused features that integrate original feature information from the feature depth direction. For example, a method of feature fusion at different levels such as a signal level, a working status level and the like may also be adopted to obtain fused features integrating the original feature information from the feature width direction. It should be understood that the above only provides exemplary fusion methods, and different data fusion methods may be adopted according to actual needs. The embodiments of the present disclosure are not limited by a specific method of data fusion.

Based on the above, according to actual needs, by extracting the working condition features, condition monitoring features and papermaking quality features related to papermaking quality from the integrated data set using various feature extraction methods, and obtaining the target feature data set based on the working condition features, condition monitoring features and papermaking quality features, the obtained target feature data set can comprehensively reflect characteristics of processing components, machine processes and/or generated paper products of the paper machine in multiple aspects. Compared with existing technical solutions that only extract a single feature and perform only a single feature extraction method, the feature data set related to papermaking quality obtained by the feature extraction method of the present disclosure can more comprehensively reflect characteristics of papermaking processing components, machine processes and generated paper products in multiple levels, multiple dimensions, and multiple aspects, which are conducive to subsequent realization of a more comprehensive and accurate evaluation of papermaking quality based on the feature data set.

At S106, for each evaluation target, a papermaking quality analysis model is established for evaluation of the corresponding evaluation target based on the target feature data set.

Based on the target feature data set related to the evaluation target obtained from S105, different types of analysis and evaluation models may be constructed for various papermaking quality evaluation applications using comprehensive methods and algorithms. It should be understood that establishment of various analysis and evaluation models related to papermaking quality described in the following embodiments of the present disclosure is only intended to be an example, rather than a specific limitation to the analysis and evaluation models. Analysis and evaluation models of other aspects related to papermaking quality may be established and configured according to actual needs, and each model may also be selected and configured according to characteristics of applications.

For example, in some embodiments, establishing a papermaking analysis model for evaluation of the evaluation target based on the target feature data set related to the evaluation target may include: for each evaluation target, establishing a quality anomaly detection model for detecting quality anomaly of the corresponding evaluation target based on the target feature data set. The quality anomaly detection model may use a variety of anomaly detection methods to detect a quality anomaly risk of the evaluation target. For example, the quality anomaly risk may include a quality anomaly risk of a target papermaking process and/or performance of related paper products for the evaluation target. The anomaly detection methods may include, but are not limited to, a K-sigma method, a boxplot method, KNN, LOF, one-class-SVM, etc., for example.

For example, in some embodiments, establishing a papermaking analysis model for evaluation of the evaluation target based on the target feature data set related to the evaluation target may include: establishing a quality level classification model for classifying a quality level of the corresponding evaluation target based on the target feature data set. The quality level classification model may use a variety of classification methods to detect and grade the evaluation target. For example, the level classification may include level classification of a target papermaking process and/or performance of related paper products for the evaluation target. The classification methods used therein may include, but are not limited to, Logistic regression, Bayes, SVM, KNN, Decision tree, Random forest, XGBoost, etc., for example.

For example, in some embodiments, establishing a papermaking analysis model for evaluation of the evaluation target based on the target feature data set related to the evaluation target may include: establishing a quality indicator prediction model for predicting quality indicators of the corresponding evaluation target based on the target feature data set related to the evaluation target. The quality indicator prediction model may use a variety of correlation analysis and regression methods to fit and predict a trend of the quality indicator of the evaluation target. For example, the quality indicator predictions may include predictions of trends in quality indicators of a target papermaking process and/or performance of related paper products for the evaluation target. The correlation analysis and regression methods used therein include, but are not limited to, Linear regression, Ridge regression, Lasso regression, SVR, Random forest, etc., for example.

Establishment and training of the above-described exemplary papermaking quality-related analysis models are based on the feature data set related to the evaluation target. With reference to the foregoing description of the present disclosure, it can be known that the feature data set related to the evaluation target is obtained based on feature extraction of the working condition data, condition monitoring data, and papermaking quality data related to the evaluation target. By combining the papermaking quality data (which may also be referred to as papermaking quality historical data), feature data may be labeled. The labeled feature data is used as an input of a corresponding analysis model, so that each analysis model can be effectively trained. Since the feature data set related to papermaking quality obtained by the aforementioned feature extraction method of the present disclosure can more comprehensively reflect characteristics of the papermaking machine or processes at multiple levels, multiple dimensions, and multiple aspects, outputs of analysis models established and trained based on the feature set will be able to reflect more comprehensive and accurate status indicators about the paper machine or processes, and thus is further beneficial to realize a more comprehensive and accurate evaluation of papermaking situations based on the outputs of the analysis models subsequently. It should be understood that the papermaking quality data may only be used in the process of establishing and training the analysis and evaluation model of the present disclosure. In the process of using the analysis and evaluation model after training, it is no longer necessary to collect papermaking quality data, but the trained papermaking quality analysis and evaluation model can be used to analyze and obtain related papermaking quality evaluation data online. With the established and trained quality analysis and evaluation model, more timely, accurate and comprehensive evaluation can be achieved, and requirements for high storage capacity and high computing power of the computing system brought about by the use of on-site image acquisition and image processing computing can be avoided.

At S107, for each evaluation target, the corresponding evaluation target is evaluated and a quality health evaluation result of the corresponding evaluation target is generated based on the papermaking quality analysis model.

For example, in some embodiments, the quality anomaly of the corresponding evaluation target may be detected and a quality anomaly detection result may be generated based on an output of the quality anomaly detection model. For example, in some embodiments, the quality level of the corresponding evaluation target may be classified and a quality level classification result may be generated based on an output of the quality level classification model. For example, in some embodiments, the quality indicator of the corresponding evaluation target may be predicted and a quality indicator prediction result may be generated based on an output of the quality indicator prediction model.

For example, in some embodiments, a quality health evaluation result for the corresponding evaluation target may be directly generated according to any one of the quality anomaly detection result, the quality level classification result, and the quality indicator prediction result. For example, in some embodiments, the quality health evaluation result for the corresponding evaluation target may also be generated according to a combination of any of the quality anomaly detection result, the quality level classification result, and the quality indicator prediction result. Thereby, the quality health status of the corresponding target can be evaluated from different perspectives such as anomaly degree, quality level, and prediction situation of various quality indicators.

At S108, a comprehensive papermaking quality evaluation result may be obtained based on at least one quality health evaluation result of at least one evaluation target. The comprehensive papermaking quality evaluation result can reflect overall quality health situation. For example, the at least one evaluation target may include at least one component of a papermaking machine. The at least one component may be a component that directly or indirectly affects the quality of papermaking, such as bearings, blades, and the like. By generating the comprehensive papermaking quality evaluation result based on the quality health evaluation result for each evaluation target, a more thorough and comprehensive evaluation of papermaking quality in all aspects of the papermaking machine can be obtained, for example, so as to facilitate better quality evaluation and quality control.

Based on the above, in the method for papermaking quality evaluation of the present disclosure, by utilizing and integrating multi-signal, multi-working condition, and multi-dimensional data related to papermaking quality of the evaluation target, and thereby extracting more comprehensive relevant features, it is possible to establish a more effective correlation model between the paper machine or paper processing status and paper product status, so as to obtain more comprehensive and accurate indicators reflecting paper quality. The papermaking quality can be comprehensively and accurately evaluated online with the effective correlation model, without the need for image acquisition of papermaking products and massive complex image processing based on image data. In this way, various situations, status and changing trends of papermaking quality can be reflected and evaluated online and in time. Moreover, it can further provide more accurate and timely warnings, alerts, feedback and optimization strategies accordingly. For example, with the method of the present disclosure, papermaking quality problems can be discovered in time, severity of papermaking quality can be classified and graded online, and a changing trend of papermaking performance can be predicted in advance according to results of the quality prediction model and then process parameters can be optimized accordingly. In addition, the papermaking quality evaluation method of the present disclosure can obtain more objective and quantitative indicators for quality evaluation and quality monitoring, realize online evaluation and control of papermaking performance, and even output more comprehensive papermaking performance criteria.

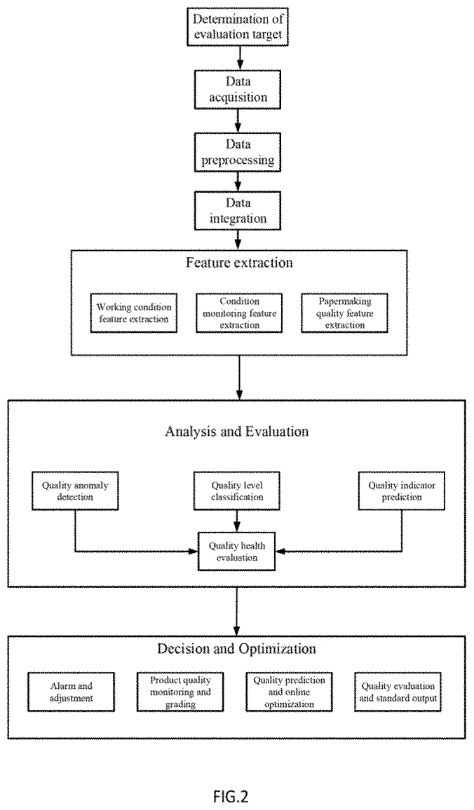

FIG. 2 exemplarily illustrates a modular framework diagram of a method or system for papermaking quality evaluation, decision and optimization according to one or more embodiments of the present disclosure. The modular framework diagram shown in FIG. 2 exemplarily shows an evaluation target determination module, a data acquisition module, a data preprocessing module, a data integration module, a feature extraction module, an analysis and evaluation module, and a decision and optimization module. Each of the above modules may be implemented in software, in hardware, or in a combination of software and hardware.

The evaluation target determination module is intended to determine a specific target for evaluation, which, for example, may perform the step at S101 in FIG. 1 to determine at least one target related to papermaking quality.

The data acquisition module is intended to acquire required data, which, for example, may perform the step at S102 in FIG. 1 for each evaluation target, to collect target working condition data, target condition monitoring data and target papermaking quality data related to the corresponding evaluation target.

The data preprocessing module is intended to process the acquired data, which, for example, may perform the step at S103 in FIG. 1 to preprocess the acquired target working condition data, target condition monitoring data and target papermaking quality data, to obtain preprocessed target working condition data, preprocessed target condition monitoring data and preprocessed target papermaking quality data.

The data integration module is intended to integrate the preprocessed data, which, for example, may perform the step at S104 in FIG. 1 to perform data integration on the preprocessed target working condition data, the preprocessed target condition monitoring data, and the preprocessed target papermaking quality data to obtain an integrated data set.

The feature extraction module is intended to perform feature extraction on the integrated data set, which, for example, may perform the step at S105 in FIG. 1 to perform feature extraction on data in the integrated data set according to types and characteristics of the data based on the integrated data set, to obtain a target feature data set. As shown in FIG. 2, the working condition feature extraction module is intended to extract features of the working condition data in the data set based on the integrated data set to obtain working condition features. The condition monitoring feature extraction module is intended to extract features of the condition monitoring data in the data set based on the integrated data set to obtain condition monitoring features. The papermaking quality feature extraction module is intended to extract features of the papermaking quality data in the data set based on the integrated data set to obtain papermaking quality features. Thus, based on the extracted working condition features, condition monitoring features, and papermaking quality features, a target feature data set related to the evaluation target may be formed for subsequent modeling, analysis, and evaluation.

The analysis and evaluation module is intended to analyze and evaluate papermaking based on the extracted feature data set. On the whole, the analysis and evaluation module may further include an analysis model establishment module and a target evaluation module. The analysis model establishment module and the target evaluation module may, for example, perform the steps at S106 and S107 in FIG. 1, respectively. For example, a papermaking quality analysis model is established for evaluation of the corresponding evaluation target based on the target feature data set; and the corresponding evaluation target is evaluated and a papermaking quality evaluation result is generated based on the papermaking quality analysis model. Operations of the analysis model establishment module and the target evaluation module are specifically described below in three exemplary aspects. It should be understood that the quality anomaly detection module, the quality level classification module and the quality indicator prediction module included in FIG. 2 are intended to respectively represent analysis modules and corresponding evaluation modules established in the three aspects of quality anomaly detection, quality level classification and quality indicator prediction. For example, the corresponding analysis models described above are respectively established, and corresponding analysis results are generated according to outputs of the corresponding analysis models, such as a quality anomaly detection result, a quality level classification result and a quality indicator prediction result. A quality health evaluation module may generate, based on at least one of the quality anomaly detection result, the quality level classification result and the quality indicator prediction result, a quality health evaluation result about papermaking that reflects the determined evaluation target from multiple perspectives and multiple dimensions.

It should be understood that a papermaking quality evaluation module (not shown in the figure) may also be included. The papermaking quality evaluation module may be configured to obtain a comprehensive papermaking quality evaluation result based on the quality health evaluation result of at least one evaluation target.

The decision and optimization module in FIG. 2 is intended to make corresponding decisions and take relevant optimization actions based on the quality health evaluation result of at least one evaluation target, or based on the obtained comprehensive papermaking quality evaluation result. It should be understood that FIG. 2 only illustrates several main aspects, and in practical applications, selection and configuration may be made according to actual needs.

For example, a quality alert is output based on the quality evaluation result, so an operator may take appropriate management and adjustment based on the quality alert.

For example, according to the quality evaluation result, monitoring situations and grading situations of papermaking condition may be output online in various forms automatically or according to the operator's call. As a result, different optimization decisions and actions may be made according to different situations.

For example, based on the quality health evaluation result, the operator may predict a trend in quality performance and accordingly perform online optimization of the paper machine's process or parameter settings.

For example, a normalized quality health result may be generated and output based on actual needs or the operator's choice, so as to acquire comprehensive normalized information for quality evaluation and quality control.

In addition, the papermaking quality evaluation system of the present disclosure may further include a self-learning and improvement module. The self-learning and improvement module may, for example, continuously collect data, connect with an operating system and cooperate with production components, and update algorithms, logic and parameters based on a self-learning mechanism, thereby realizing automatic improvement of the system.

Based on the above, in the method and system for papermaking quality evaluation of the present disclosure, by utilizing and integrating multi-signal, multi-working condition, and multi-dimensional data related to papermaking quality of the evaluation target, and thereby extracting more comprehensive relevant features, it is possible to establish a more effective correlation model between the paper machine or paper processing status and paper product status, so as to obtain more comprehensive and accurate indicators reflecting paper quality. The papermaking quality can be comprehensively and accurately evaluated online with the effective correlation model, without the need for image acquisition of papermaking products and massive complex image processing based on image data. In this way, various situations, status and changing trends of papermaking quality can be reflected and evaluated online and in time. Moreover, it can further provide more accurate and timely warnings, alerts, feedback and optimization strategies accordingly. For example, with the method and system of the present disclosure, papermaking quality problems can be discovered in time, severity of papermaking quality can be classified and graded online, and a changing trend in papermaking performance can be predicted in advance according to results of the quality prediction model and process parameters can be optimized accordingly. In addition, the papermaking quality evaluation method and system of the present disclosure can obtain more objective and quantitative indicators for quality evaluation and quality monitoring, realize online evaluation and control of papermaking performance, and even output more comprehensive papermaking performance criteria.

In addition, with the method and system of the present disclosure, it is possible to monitor, evaluate, control and optimize the papermaking process, status and performance in a continuous closed loop, thereby greatly improving the papermaking quality evaluation, control capability and solution capability. Furthermore, by processing and modeling acquired data based on data or machine learning, it is possible to support evaluation and optimization of papermaking quality in a data-driven, knowledge-driven digitalized and intelligent manner.

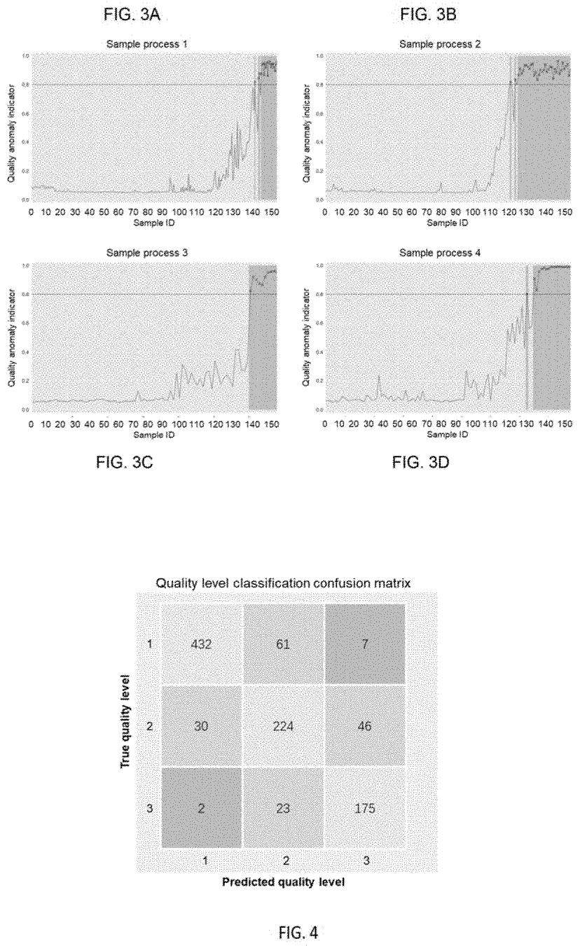

FIGS. 3A-3D illustrate a schematic diagram of an exemplary quality anomaly detection according to the method and system of the present disclosure, where quality anomaly degree trend diagrams of four papermaking processes are exemplarily shown. In the example of FIGS. 3A-3D, data sampling is performed at a sampling frequency of 100 kHz, for example. In the four graphs of FIG. 3A-3D, the horizontal axis represents a sampling ID, the vertical axis is a normalized value of a quality anomaly indicator, and the dotted line corresponds to a threshold for quality anomaly detection. In the figure, detected abnormal situations are prompted, referring to distinguishing marks above the threshold dotted line in the figure.

FIG. 4 exemplarily illustrates a confusion matrix of quality level classification results for a plurality of papermaking processes during testing. The predicted quality levels on the horizontal axis are results obtained by the quality level classification model in the papermaking quality evaluation method and system of the present disclosure, and the vertical axis represents true quality level classification results. Diagonal data of the matrix in the figure represents the number of samples with the same predicted results and true results. It can be seen that the papermaking quality evaluation method and system of the present disclosure can establish an appropriate analysis model and generate analysis and evaluation results with high accuracy.

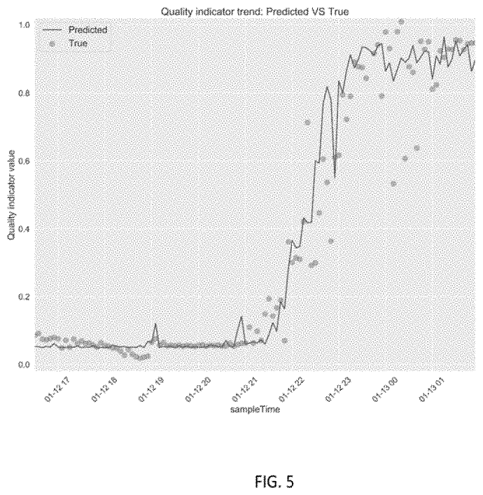

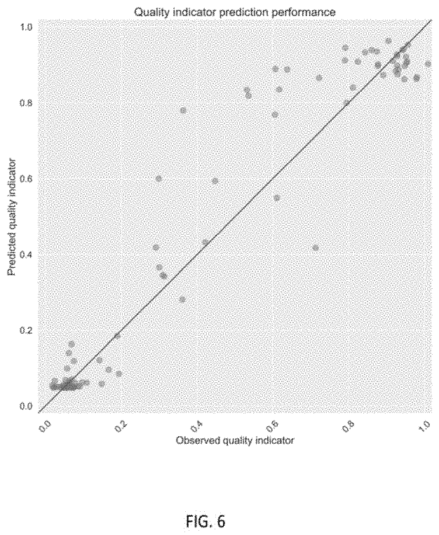

FIG. 5 exemplarily illustrates prediction results of a quality indicator for various papermaking processes during testing. In the example of FIG. 5, the horizontal axis represents sampling time, and the vertical axis represents normalized values of the quality indicator. The line represents fitting results of quality indicator predictions obtained by the quality indicator prediction model in the papermaking quality evaluation method and system of the present disclosure, and the circle dots represent true quality indicator results. It can also be seen from FIG. 5 that, with the quality indicator prediction model in the method and system for paper quality evaluation of the present disclosure, the quality prediction indicator obtained according to extracted features can well show status and trend of papermaking quality. Therefore, the papermaking quality evaluation method of the present disclosure may also be used to predict a papermaking quality situation in advance. It can be seen from the comparison that the papermaking quality evaluation method and system of the present disclosure can establish an appropriate analysis model and generate analysis and evaluation results with high accuracy. A fitting and regression performance of the quality indicator predictions is shown in FIG. 6.

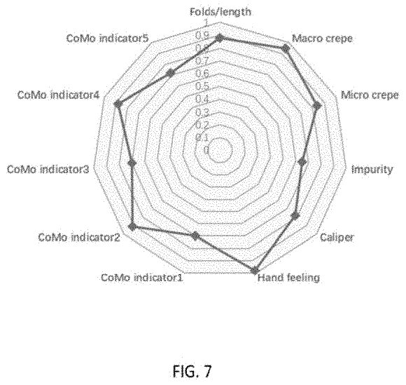

FIG. 7 illustrates a radar plot of an exemplary paper quality health evaluation drawn based on partial quality health evaluation results obtained using the papermaking quality evaluation method and system of the present disclosure. In addition to paper detection indicators (e.g., crepe frequency (folds/length), large crepe ratio (macro crepe), small crepe ratio (macro crepe), crepe impurity, caliper, hand feeling), the radar plot also incorporates quality health evaluation indicators from model analysis (e.g., CoMo indicators 1-5 in the figure). Values from 0 to 1 represent a heath level (e.g., the value “1” is the best heath level, the value “0” is the lowest heath level). It can be seen that based on the comprehensive evaluation of the papermaking quality evaluation method and system of the present disclosure, more comprehensive, objective and quantitative indicators can be obtained. These indicators may be used to evaluate, control and optimize papermaking processes, and even output more comprehensive papermaking performance criteria.

According to another aspect of the present invention, there is also provided a non-volatile computer-readable storage medium on which computer-readable instructions are stored, and when the instructions are executed by a computer, the aforementioned method may be performed, and has the functions described above.

A program portion of the technology may be considered as a “product” or “article of manufacture” in the form of executable code and/or related data, which is embodied or implemented via a computer-readable medium. Tangible, persistent storage media may include memory or storage used by any computer, processor, or similar device or associated module, for example, various semiconductor memories, tape drives, disk drives, or any device capable of providing storage functionality for software.

All or part of the software may sometimes communicate over a network, such as the Internet or other communication network. Such communications may load software from one computer device or processor to another. Therefore, another medium capable of transmitting software elements may also be used as a physical connection between local devices, such as light waves, radio waves, electromagnetic waves, etc., which are propagated through cables, optical cables, or air. A physical medium used for carrying waves, such as a cable, wireless connection, or fiber optic cable, etc., may also be considered as a medium that carries the software. Unless limited to tangible “storage” media herein, other terms referring to computer or machine “readable media” refer to media that participate in execution of any instructions by a processor.

This application uses specific terms to describe the embodiments of the present application. “First/second embodiment”, “an embodiment”, and/or “some embodiments” is intended to mean a certain feature, structure or characteristic associated with at least one embodiment of the present application. Therefore, it should be emphasized and noted that two or more references to “an embodiment” or “one embodiment” or “an alternative embodiment” in different places in this specification are not necessarily referring to the same embodiment. Furthermore, certain features, structures or characteristics of one or more embodiments of the present application may be combined as appropriate.

Furthermore, those skilled in the art will appreciate that aspects of the present application may be illustrated and described in several patentable categories or situations, including any new and useful process, machine, product, or combination of matter, or any new and useful improvement to them. Accordingly, various aspects of the present application may be performed entirely by hardware, entirely by software (including firmware, resident software, microcode, etc.), or by a combination of hardware and software. The above hardware or software may be referred to as a “data block”, “module”, “engine”, “unit”, “component” or “system”. Furthermore, aspects of the present application may be embodied as a computer product comprising computer readable program code embodied in one or more computer readable media.

The present disclosure has been described above with reference to specific embodiments. However, one of ordinary skill in the art will appreciate that various modifications and changes may be made therein without departing from the broad spirit and scope of the present disclosure as set forth in the appended claims.

What is claimed is:

1. A method for evaluating papermaking quality comprising:
determining at least one evaluation target related to the papermaking quality;
for each evaluation target:
acquiring target working condition data, target condition monitoring data and target papermaking quality data related to the corresponding evaluation target;
preprocessing the acquired target working condition data, target condition monitoring data and target papermaking quality data to obtain preprocessed target working condition data, preprocessed target condition monitoring data and preprocessed target papermaking quality data;
performing data integration on the preprocessed target working condition data, the preprocessed target condition monitoring data, and the preprocessed target papermaking quality data to obtain an integrated data set;
performing feature extraction on data in the integrated data set according to types and characteristics of the data based on the integrated data set to obtain a target feature data set;
establishing a paper quality analysis model for evaluation of the corresponding evaluation target based on the target feature data set; and
evaluating the corresponding evaluation target and generating a quality health evaluation result of the corresponding evaluation target based on the papermaking quality analysis model;
obtaining a comprehensive papermaking quality evaluation result based on the quality health evaluation result of the at least one evaluation target.

2. The method for evaluating papermaking quality according to claim 1, wherein for each evaluation target, the performing feature extraction on data in the integrated data set according to types and characteristics of the data based on the integrated data set to obtain a target feature data set comprises:
extracting features of working condition data in the integrated data set to obtain working condition features;
extracting features of condition monitoring data in the integrated data set to obtain condition monitoring features;
extracting features of papermaking quality data in the integrated data set to obtain papermaking quality features;
obtaining the target feature data set based on the working condition features, the condition monitoring features and the papermaking quality features.

3. The method for evaluating papermaking quality according to claim 2, wherein for each evaluation target, the obtaining the target feature data set based on the working condition features, the condition monitoring features and the papermaking quality features comprises:
obtaining fused feature data by feature fusion processing based on the working condition features, the condition monitoring features and the papermaking quality features, and generating the target feature data set based on the fused feature data.

4. The method for evaluating papermaking quality according to claim 1, wherein, for each evaluation target, the establishing a paper quality analysis model for evaluation of the corresponding evaluation target based on the target feature data set comprises:
establishing a quality anomaly detection model for detecting quality anomaly of the corresponding evaluation target based on the target feature data set;
establishing a quality level classification model for classifying a quality level of the corresponding evaluation target based on the target feature data set; and
establishing a quality indicator prediction model for predicting quality indicators of the corresponding evaluation target based on the target feature data set.

5. The method for evaluating papermaking quality according to claim 4, wherein for each evaluation target, the evaluating the corresponding evaluation target and generating a quality health evaluation result of the corresponding evaluation target based on the papermaking quality analysis model includes:
detecting the quality anomaly of the corresponding evaluation target and generating a quality anomaly detection result based on an output of the quality anomaly detection model;
classifying the quality level of the corresponding evaluation target and generating a quality level classification result based on an output of the quality level classification model;
predicting the quality indicators of the corresponding evaluation target and generating a quality indicator prediction result based on an output of the quality indicator prediction model; and
generating the quality health evaluation result of the corresponding evaluation target based on at least one of the quality anomaly detection result, the quality level classification result, and the quality indicator prediction result.

6. The method for evaluating papermaking quality according to claim 1, wherein for each evaluation target, the preprocessing the acquired target working condition data, target condition monitoring data and target papermaking quality data comprises performing at least one of data deduplication processing, data noise reduction processing, data encoding processing, and data filtering processing.

7. The method for evaluating papermaking quality according to claim 1, wherein for each evaluation target, the performing data integration on the preprocessed target working condition data, the preprocessed target condition monitoring data, and the preprocessed target papermaking quality data comprises:
performing at least one of synchronization, alignment and correction processing on the preprocessed target working condition data, the preprocessed target condition monitoring data, and the preprocessed target papermaking quality data.

8. The method for evaluating papermaking quality according to claim 1, wherein the at least one evaluation target includes at least one component of a papermaking machine.

9. A non-transient computer-readable storage medium having computer-readable instructions stored thereon, which, when executed by a computer, perform the method of claim 1.

10. A system for evaluating papermaking quality comprising:
an evaluation target determination module configured to determine at least one evaluation target related to papermaking quality;
a data acquisition module configured to, for each evaluation target, acquire target working condition data, target condition monitoring data and target papermaking quality data related to the corresponding evaluation target;
a data preprocessing module configured to, for each evaluation target, preprocess the acquired target working condition data, target condition monitoring data and target papermaking quality data to obtain preprocessed target working condition data, preprocessed target condition monitoring data and preprocessed target papermaking quality data;
a data integration module configured to, for each evaluation target, perform data integration on the preprocessed target working condition data, the preprocessed target condition monitoring data, and the preprocessed target papermaking quality data to obtain an integrated data set;
a feature extraction module configured to, for each evaluation target, perform feature extraction on data in the integrated data set according to types and characteristics of the data based on the integrated data set to obtain a target feature data set;
an analysis model establishment module configured to, for each evaluation target, establish a paper quality analysis model for evaluation of the corresponding evaluation target based on the target feature data set;
a target evaluation module configured to, for each evaluation target, evaluate the corresponding evaluation target and generate a quality health evaluation result of the corresponding evaluation target based on the papermaking quality analysis model; and
a papermaking quality evaluation module configured to obtain a comprehensive papermaking quality evaluation result based on the quality health evaluation result of the at least one evaluation target.
